(19) United States
(12) Patent Application Publication
(10) Pub. No.: US 2016/0000720 A1
(43) Pub. Date: Jan. 7, 2016

(54) Pharmaceutical compositions comprising Tadalafil

(71) Applicant: AUROBINDO PHARMA LIMITED, Hyderabad, Andhra Pradesh (IN)

(72) Inventors: Krishna Murthy Bhavanasi, Hyderabad (IN); Nagaprasad Vishnubhotla, Hyderabad (IN); Ramesh Manchala, Hyderabad (IN); Sivakumaran Meenakshisunderam, Hyderabad (IN)

(21) Appl. No.: 14/767,858

(22) PCT Filed: Feb. 7, 2014
(86) PCT No.: PCT/IB2014/000133

§ 371 Date: Aug. 13, 2015

(30) Foreign Application Priority Data
Feb. 14, 2013 (IN) 658/CHE/2013

(51) Int. Cl. A61K 9/20; B29C 37/00; B29C 47/00; A61K 31/4985; A61K 9/28

(52) U.S. Cl. CPC A61K 9/2077; A61K 31/4985; A61K 9/2095; A61K 9/2893; A61K 9/2027; A61K 9/2054; A61K 9/2031; A61K 9/2013; A61K 9/2018; A61K 9/2009; B29C 47/004; B29C 37/0025; B29K 2023/06; B29L 2031/772

(57) ABSTRACT

Pharmaceutical compositions comprising tadalafil or a pharmaceutically acceptable salt thereof are provided. The present invention also relates to a process for preparation of pharmaceutical compositions comprising tadalafil or a pharmaceutically acceptable salt thereof. The present invention also relates to method of administering the compositions comprising tadalafil in a subject in need thereof.

### FIELD OF THE INVENTION

The present invention relates to pharmaceutical compositions comprising tadalafil or a pharmaceutically acceptable salt thereof. The present invention also relates to a process for preparation of pharmaceutical compositions comprising tadalafil or a pharmaceutically acceptable salt thereof. The present invention also relates to method of administering the compositions comprising tadalafil in a subject in need thereof.

### BACKGROUND OF THE INVENTION

Tadalafil is chemically known as (6R,12aR)-6-(1,3-benzodioxol-5-yl)-2,3,6,7,12,12a hexa hydro-2-methylpyrazino[1′,2′:1,6]pyrido[3,4-b]indole-1,4-dione and is disclosed in U.S. Pat. No. 5,859,006. Tadalafil belongs to the group of PDE-V (phosphodiesterase V) inhibitors and is used in the treatment of erectile dysfunction and also in the treatment of pulmonary arterial hypertension. Tadalafil is marketed in the form of tablets under the trade names Cialis® and Adcirca® in the United States.

U.S. Pat. No. 6,140,329 disclosed a method for the treatment of erectile dysfunction using tadalafil. U.S. Pat. No. 6,943,166 discloses a method of treating sexual dysfunction by administering a unit dose containing about 1 to about 20 mg of tadalafil.

Tadalafil is a solid that is understood to be practically insoluble in water and only very slightly soluble in some organic solvents. According to PCT publication WO 01/08687, tadalafil is only soluble in water in a concentration up to 2 μg/ml. The extremely limited solubility of tadalafil poses many major difficulties and challenges when formulating a dosage form that demonstrates acceptable bioavailability.

There are several prior art references which disclose various attempts to improve the solubility of tadalafil.

U.S. Pat. No. 5,985,326 describes a method of production of a solid dispersion, which contains a sparingly soluble active substance. The solubility of the active substance is said to be improved by coprecipitation. Tests on release of the active substance tadalafil have shown, however, that tablets that contain the coprecipitates release the active substance more slowly than tablets containing pure active substance. Moreover, in coprecipitates there are also proportions of tadalafil particles that are not embedded in the solid material, but are free. These free particles dissolve more quickly than the particles embedded in the coprecipitate. This may possibly lead to undesirable, bimodal release of the tadalafil. Furthermore, coprecipitates are not easily reproducible, i.e. large-scale production is complicated.

Another possibility for improving the solubility of sparingly soluble active substances is to increase the surface area of the particles of active substance by grinding or micronizing, as disclosed in U.S. Pat. No. 6,821,975 or U.S. Pat. No. 7,182,958. U.S. Pat. No. 6,821,975 discloses a free drug particulate form of tadalafil, comprising particles of tadalafil wherein at least 90% of the particles have a particle size of less than about 40 microns. U.S. Pat. No. 7,182,958 discloses a pharmaceutical formulation comprising tadalafil in “free drug” form comprising particles wherein at least 90% of the particles of the said compound have a particle size of less than about 40 microns in admixture with a diluent, lubricant, a hydrophilic binder, and a disintegrant.

The desired solubility or release could be achieved by reducing the tadalafil particle size to below 40 microns. Grinding or micronizing the active substance has several disadvantages. Micronized particles tend to form agglomerates. This results in particle sizes that are difficult to define, and accordingly solubility which is difficult to define. A possible additional electrostatic charge on the active substance also has an adverse effect on processability. Another possible disadvantage is poor flowability of the ground active substance; especially if tablets are to be compressed or capsules are to be filled, further processing steps, e.g. granulation, are necessary. Although the particles are small, it is often necessary to add a lot of surfactant to obtain adequate solubility.

U.S. Pat. No. 7,417,044 discloses particulate tadalafil wherein at least 90% of the particles have a particle size of about 200 to about 600 microns. US patent publication 2007/0031349 discloses a pharmaceutical composition comprising a rapid release component comprising at least on PDE5 inhibitor and an orally disintegrating carrier, wherein the rapid release component results in a therapeutically effective blood concentration of the PDE5 inhibitor in about 1 minute to about 20 minutes. US patent publication 2007/0098804 discloses a solid particulate tadalafil having a bimodal size distribution. US patent publication 2007/0104792 discloses a stable nanoparticulate tadalafil or a salt or derivative thereof, composition comprising: (a) particles of tadalafil having an effective average particle size of less than about 2000 nm; and (b) at least one surface stabilizer. US patent publication 2009/0098211 discloses a solid pharmaceutical dosage form comprising tadalafil and starch, wherein the tadalafil has a particle size distribution such that d(0.9) is greater than or equal to 40 μm, and wherein the weight ratio of starch to tadalafil is about 4.5:1 or more.

US patent publication 2008/0009502 discloses a solid composite comprising tadalafil and at least one carrier wherein at least about 85 wt % of the tadalafil is in intimate association with the at least one carrier. US 2010/0099687 discloses a method of making a solid composite of tadalafil and at least one carrier comprising the steps of: (a) combining the tadalafil, the at least one carrier and at least one solvent to form a solution and (b) removing the solvent from the combination to obtain the solid composite. US 2011/0263606 discloses a pharmaceutical oral dosage form comprising tadalafil prepared by: dissolving a therapeutically effective amount of tadalafil in a liquid medium containing one or more solvents and at least one tadalafil solubility enhancer; and drying the mixture.

PCT publication WO 01/08687 discloses soft capsules made of gelatin, which are filled with tadalafil solution. The solvent for tadalafil is a mixture of PEG (polyethylene glycol) 400 NF LA and polypropylene glycol. Alternatively the capsules can be filled with a tadalafil suspension. Soft capsules made of gelatin are also known from PCT publication WO 00/66099, using exclusively PEG 400 NF as solvent for the active substance. However, filing in soft gelatin capsules is a complicated process as special machines and strict climate-controlled manufacturing rooms are required. In contrast, hard capsules can be filled relatively easily with standard capsule filling machines, equipped with feed systems for semi-solid substances instead of powder feed systems. Apart from gelatin as capsule material, it is also possible to use cellulose-based materials. PCT publication WO 2010/115886 A1 discloses an adsorbate comprising an active pharmaceutical ingredient (API) being practically insoluble in water associated with a particulate and/or porous carrier, wherein the adsorbate is prepared by using a non-polar solvent or a mixture of non-polar solvents, and wherein essentially no API is in the form of precipitates, particles or crystals. PCT publication WO 2011/012217 A2 discloses a co-precipitate comprising a phosphodiesterase-5 inhibitor (PDE-5-inhibitor) and a pharmaceutically compatible copolymer carrier comprising two or more different acrylic acid derivatives. PCT publication WO 2012/085927 A2 discloses a solid dispersion comprising tadalafil or its pharmaceutically acceptable salts thereof and optionally other pharmaceutically acceptable excipients, wherein said tadalafil is substantially in crystalline state. PCT publication WO 2012/095151 A1 discloses a process for the production of pharmaceutical composition comprising the steps of (a) preparing a solution of tadalafil in organic solvent, (b) carrying up tadalafil onto a water-insoluble polymer mixed with a diluent by fluid bed granulation, (c) mixing the granulation with one or more further excipients. WO 2012/107090 A1 discloses a co-granulate comprising tadalafil and a superdisintegrant selected from a group comprising: a modified cellulose; a cross-linked polymer; and/or a modified starch. PCT publications WO 2012/107541 A1 and WO 2012/107092 A1 disclose a solid pharmaceutical composition comprising a co-granulate of tadalafil with beta-cyclodextrin in molar ratio higher than 1:1 and at least one pharmaceutically acceptable extragranular excipient. IN 2968/DEL/2005 discloses micronized tadalafil having a particle size of about 300 to 500 microns.

US patent publication 2010/0179159 discloses a method for the production of a pharmaceutical containing tadalafil, characterized in that Tadalafil is mixed with one or more suitable excipients and is heated to a temperature from approximately 100° C. to approximately 200° C., preferably approximately 150° C. to approximately 200° C. and in particular approximately 200° C. The said publication further discloses that tadalafil could be processed very well at elevated temperatures as a solid or semi-solid solution. With solid and semi-solid solutions, basically there are no agglomerates, as the active substance is distributed in a suitable polymer as a molecular dispersion. Solid solutions are characterized in that they are transparent and solid. Semi-solid solutions are characterized in that they are transparent and paste-like, i.e. not liquid. The term transparent means that the tadalafil, which is usually in the form of white powder, is dissolved in the suitable excipient. The melt granulation process disclosed in US patent publication 2010/0179159 is a promising method to develop tadalafil composition with improved bioavailability. However, due to the exposure of tadalafil to higher temperatures, there is a chance of degradation of tadalafil leading to stability problems. Secondly, due to the formation of tadalafil in the form of solid or semi solid solution, there is a chance of crystalline form getting converted to amorphous form. As per the literature, crystalline form of any drug is more stable than amorphous form.

The above prior art references disclose various techniques to improve the solubility of tadalafil like solid dispersion, co-precipitation, particle size reduction, adsorption, co-granulation, melt extrusion at high temperatures. Still, there exists a need to develop a pharmaceutical composition comprising tadalafil which shows better/comparable stability and bioavailability w.r.t marketed formulation. The problem to be solved by the present invention was therefore to provide a method for the production of a pharmaceutical containing tadalafil, in which the aforementioned disadvantages and in particular the difficult solubility of tadalafil are avoided or overcome.

The inventors of present invention have developed compositions comprising tadalafil which shows better/comparable stability and in-vitro dissolution w.r.t marketed formulation. The inventors have developed a melt granulation process for preparing composition comprising tadalafil in which tadalafil is exposed to a temperature below 95° C., which prevents the drug from decomposition. Further, there is no formation of solid or semisolid solution of tadalafil which results in maintaining tadalafil in crystalline form. Furthermore, the process according to the present invention involves the step of blending the drug with melted excipients followed by hot melt extrusion technology to maintain uniform distribution of excipients and the drug. This process also helps in maintaining less temperature in the extruder, which ultimately helps in reduction of hard lumps and also prevents the drug from decomposition.

### OBJECTIVE OF THE INVENTION

The objective of the present invention is to provide a pharmaceutical composition comprising tadalafil or a pharmaceutically acceptable salt thereof and one or more pharmaceutically acceptable excipients, process of preparation, and use thereof.

### SUMMARY OF THE INVENTION

An aspect of the present invention relates to a pharmaceutical composition comprising tadalafil or a pharmaceutically acceptable salt thereof and one or more pharmaceutically acceptable excipients.

Another aspect of the present invention relates to a pharmaceutical composition comprising tadalafil or a pharmaceutically acceptable salt thereof having particle size D₉₀ more than about 40 microns, and one or more pharmaceutically acceptable excipients.

Yet another aspect of the present invention relates to a process for preparing a pharmaceutical composition comprising tadalafil, which comprises the steps of:

(i) blending tadalafil with one or more pharmaceutically acceptable excipients,

(ii) melt extruding the material of step (i) using a hot melt extruder by maintaining the temperature below about 100° C. in different zones of the said melt extruder,

(iii) blending the extrudates of step (ii) with one or more pharmaceutically acceptable excipients,

(iv) formulating the blend of step (iii) into a suitable dosage form, and

(v) optionally coating the dosage form thus obtained in step (iv).

An aspect of the present invention relates to a method of treating one or more of disease(s)/disorder(s) selected from sexual dysfunction such as male erectile dysfunction or female sexual arousal disorder, benign prostatic hyperplasia and pulmonary hypertension, by administering a pharmaceutical composition comprising tadalafil or a pharmaceutically acceptable salt thereof and one or more pharmaceutically acceptable excipients.

### DETAILED DESCRIPTION OF THE INVENTION

The term “particle size” unless indicated otherwise in the specification relates to particles of tadalafil free base as well as pharmaceutically acceptable salt, amorphous or crystalline, anhydrous, esters, or isomer or derivative, hydrate, prodrug or solvates thereof. Tadalafil with specific “particle size” and distribution, or surface area would provide a fast dissolution of the active ingredient, would be easy to prepare and stable while maintaining the beneficial properties with respect to fast solubility and bioavailability.

The term “composition” or “formulation” or “dosage form” or “medicinal preparation” as used herein synonymously include solid dosage forms such as granules, multiunit particulate systems (MUPS), pellets, spheres, tablets, capsules, mini-tablets, beads, particles and the like; meant for oral administration.

The term “therapeutically effective amount” is defined to mean the amount or quantity of the active drug (e.g. tadalafil), which is sufficient to elicit an appreciable biological response when administered to the patient.

In accordance with the present invention, the term “tadalafil” unless indicated otherwise in the entire specification refers to tadalafil in the form of free base or its pharmaceutically acceptable salt, amorphous, various polymorphs or any isomer or derivative, hydrate or solvate, prodrug or combinations thereof. Preferably tadalafil is in the form of free base. The amount of tadalafil used may be in the range of 1-40% by weight of the composition, preferably in the range of 1-20%. In an embodiment, the polymorphic form is substantially retained in the formulation at the initial stage and during stability charge.

The term “excipient” means a pharmacologically inactive component such as a diluent, disintegrant, carrier, or the like. The excipients that are useful in preparing a pharmaceutical composition are generally safe, non-toxic and are acceptable for veterinary as well as human pharmaceutical use. Reference to an excipient includes both one and more than one such excipient. The phrase “medicinal package” unless indicated otherwise in the entire specification refers to bottle or blister pack or pouch or any corresponding packing known to a person skilled in the art in which the dosage form or the medicinal preparation is packed.

The term “desiccant” unless indicated otherwise in the entire specification refers to a substance used to remove/suppress/decrease the odor/smell or to absorb moisture which prevents degradation/decomposition of the active agent(s). The desiccant may be placed in the internal space of the medicinal package, irrespective of any particular limit, so long as the amount is sufficient to remove the odorous material, that is, sufficient to suppress or reduce the smell. The amount of the desiccant can vary depending on kind or shape of the desiccant, distance from the medicinal preparation capable of giving out smells, amount of the compound giving out smells, type of formulation, volume of the space where the medicinal preparation and the desiccant are placed, amount of the existing or produced odorous material, preservation condition of the medicinal package. In another embodiment, the composition comprising tadalafil according to the present invention was found to be stable after storage at 40° C./75% RH for at least three months.

As used in this specification, the singular forms “a”, “an”, and “the” include plural references unless the context clearly dictates otherwise. Thus for example, a reference to “a process” includes one or more process, and/or steps of the type described herein and/or which will become apparent to those persons skilled in the art upon reading this disclosure and so forth.

In an embodiment, the present invention relates to a pharmaceutical composition comprising tadalafil or a pharmaceutically acceptable salt thereof and one or more pharmaceutically acceptable excipients.

In another embodiment, the present invention relates to a pharmaceutical composition comprising tadalafil or a pharmaceutically acceptable salt thereof having particle size D₉₀ more than about 40 microns and one or more pharmaceutically acceptable excipients. In another embodiment, the present invention relates to a pharmaceutical composition comprising tadalafil or a pharmaceutically acceptable salt thereof having particle size D₉₀ in the range of between about 42 microns to less than about 200 microns.

The term “D90” unless indicated otherwise in the specification is defined as the size value corresponding to cumulative size distribution at 90%, which represents the size of particles below which 90% of the sample lies. The known particle size analysis methods are suitable for determining the particle size, for example, particle size measurement using light-scattering methods such as by Malvern or Horiba.

In an embodiment, the present invention relates to a pharmaceutical composition comprising tadalafil or a pharmaceutically acceptable salt thereof and one or more pharmaceutically acceptable excipients wherein the composition is prepared by melt extrusion process.

In an embodiment, the present invention relates to a pharmaceutical composition comprising tadalafil or a pharmaceutically acceptable salt thereof and one or more pharmaceutically acceptable excipients wherein the composition is prepared by melt extrusion process by maintaining the temperature below 100° C., preferably below 95° C. in different zones of the melt extruder.

In another embodiment, the process for forming extrudates according to the present invention takes place in hot melt extrusion equipment, in which the operating conditions involve setting the parameters of the hot melt extruder such as feed rate, screw speed, zone temperatures, die temperature, melt temperature, chiller temperature and chill roll speed in order to achieve the end product in the form of a composition.

“Pharmaceutically acceptable excipient/s” are the components added to pharmaceutical formulation to facilitate manufacture, enhance stability, control release, enhance product characteristics, enhance bioavailability, enhance patient acceptability, etc. Pharmaceutically acceptable excipients includes, but not limited to, water soluble polymers, diluents or fillers, binders, disintegrants, antioxidants, sugars, lubricants, glidants, compression aids, colors, sweeteners, preservatives, surfactants, suspending agents, dispersing agents, film formers, flavors, printing inks, and the like, and mixtures thereof.

The pharmaceutically acceptable water soluble polymers according to the present invention are selected from but not limited to group comprising to homopolymers and copolymers of N-vinyl lactams, especially homopolymers and copolymers of N-vinyl pyrrolidone, e.g. polyvinylpyrrolidone, copolymers of N-vinyl pyrrolidone and vinyl acetate e.g. copovidone, methyl cellulose, hydroxypropyl cellulose, hydroxypropylmethyl cellulose, cellulose phthalates or succinates, cellulose acetate phthalate hydroxypropyl methylcellulose phthalate, polyethylene oxide, polyacrylates, methyl methacrylate, butyl methacrylate, Polyvinyl caprolactam-polyvinyl acetate-polyethylene glycol graft; polyethylene glycol, and the like, and mixtures thereof. The water soluble polymer may be used in the range of about 1-20% by weight of the composition.

Diluents or fillers increase the bulk of the composition. Diluents or fillers according to the present invention are selected from but not limited to group comprising sugars such as lactose, sucrose, dextrose; sugar alcohols such as mannitol, sorbitol, xylitol, lactitol; Starlac® (co-processed mixture of Starch and lactose), Microcelac® (co-processed mixture of microcrystalline cellulose and lactose), starch, corn starch, modified starches, pregelatinized starch, dibasic calcium phosphate, tribasic calcium phosphate, powdered cellulose, microcrystalline cellulose, silicified microcrystalline cellulose, and the like, and mixtures thereof. The diluent may be used in the range of 5-95% by weight of the dosage form. Binders hold the ingredients in the composition together. Exemplary binders are selected from but not limited to group comprising, cellulose and its derivatives including, ethyl cellulose, hydroxypropyl cellulose, hydroxypropyl methylcellulose, methylcellulose and hydroxyethyl cellulose, carboxymethyl cellulose; starch and its derivatives; polyvinylalcohol, polyvinyl alcohol based compositions such as Opadry® HP, Opadry® II white or Instacoat® and the like; hydrocolloids; sugars; polyvinyl pyrrolidone, copovidone, methacrylic acid copolymers, and the like, and mixtures thereof. The binder may be used in the range of 1-20% by weight of the dosage form. Disintegrants according to the present invention are selected from but not limited to group comprising, water swellable substances, for example, cellulose and its derivatives including low-substituted hydroxypropyl cellulose; cross-linked polyvinylpyrrolidone; cross-linked sodium carboxymethylcellulose, cross-linked calcium carboxymethylcellulose, sodium carboxymethylcellulose, calcium carboxy methylcellulose, microcrystalline cellulose; sodium starch glycolate; ion-exchange resins; starch and modified starches including pregelatinized starch; formalin-casein; alginates, gums, and the like, and mixtures thereof. The disintegrant may be used in the range of 1-30% by weight of the dosage form.

Surfactants are compounds which are capable of improving the wetting of the drug and/or enhancing the dissolution. The surfactants can be selected from hydrophilic surfactants or lipophilic surfactants and mixtures thereof. The surfactants can be anionic, nonionic, cationic, and zwitterionic surfactants. Surfactants according to the present invention are selected from but not limited to group comprising, polyoxyethylene alkylaryl ethers such as polyoxyethylene lauryl ether, polyoxyethylene cetyl ether, polyoxyethylene stearyl ether; polyethylene glycol; polyethylene glycol fatty acid esters such as PEG monolaurate, PEG dilaurate, Polyethylene glycol 660 12-hydroxyl Stearate Ph. Eur. or Polyoxyl 15 hydroxystearate NF (Solutol HS 15), PEG distearate, PEG dioleate; polyoxyethylene sorbitan fatty acid ester such as polysorbate 40, polysorbate 60, polysorbate 80; sorbitan fatty acid mono esters such as sorbitan monolaurate, sorbitan monooleate, sorbitan sesquioleate, sorbitan trioleate, polyoxyethylene castor oil derivates such as polyoxyl castor oil, polyoxyl hydrogenated castor oil, sodium lauryl sulphate, monooleate, monolaurate, monopalmitate, monostearate, sodium dioctyl sulfosuccinate (DOSS), lecithin, stearylic alcohol, cetostearylic alcohol, cholesterol, polyoxyethylene ricin oil, polyoxyethylene, fatty acid glycerides, poloxamer, Cremophore RH 40, and the like, and mixtures thereof. The surfactant may be used in the range of 0.001-5% by weight of the dosage form. Lubricants and glidants aid in the processing of powder materials. Exemplary lubricants are selected from but not limited to group comprising, calcium stearate, glyceryl behenate, magnesium stearate, mineral oil, polyethylene glycol, sodium stearyl fumarate, stearic acid, fumaric acid, talc, vegetable oil, zinc stearate, and the like, or mixtures thereof. The lubricant may be used in the range of 0.01-5% by weight of the dosage form. Exemplary glidants include, but not limited to, talc, silicon dioxide, silicic acid, corn starch and the like, and mixtures thereof. The glidant may be used in the range of 0.01-5% by weight of the dosage form.

The dosage form according to the present invention may be uncoated or optionally coated with functional coating, film coating, moisture barrier coating or a protective coating composition. The coating composition mainly comprises of film forming polymers and one or more of plasticizers, opacifier, surfactant, anti tacking agents, coloring agent and the like. The coating according to the present invention is applied by solubilising or suspending the excipients in solvents such as isopropyl alcohol, water, acetone, ethanol, methylene chloride, 0.1N HCl and the like or mixtures thereof. Varieties of commercially available cellulosic polymers exist and may include, for example, Spectracel® HPMC compositions (available from Sensient Technologies). Further, other commercially available coating materials are available marketed under the brand name Opadry® for example Opadry II Gray which contains: lactose monohydrate NF, hypromellose type 2910 USP, titanium dioxide USP, triacetin USP, and iron oxide black JPE; Opadry II Pink which contains: hypromellose type 2910 USP, titanium dioxide USP, lactose monohydrate NF, polyethylene glycol 3350 NF, triacetin USP, and FD&C Red #40; Opadry II Blue which contains: hypromellose type 2910 USP, lactose monohydrate NF, FD&C Blue #1, polyethylene glycol 3350 NF, FD&C Blue #2, titanium dioxide USP, triacetin USP, and D&C Yellow #10; Opadry II Yellow which contains: hypromellose type 2910 USP, lactose monohydrate NF, titanium dioxide USP, iron oxide yellow NF, polyethylene glycol 3350 NF, and triacetin USP; Opadry II Purple which contains: hypromellose type 2910 USP, lactose monohydrate NF, titanium dioxide USP, D&C Red #27, polyethylene glycol 3350 NF, triacetin USP, and FD&C Blue #1 and the like.

In an embodiment, the compositions of the present invention may additionally comprise of a colorant in order to produce a desirable color. Colors known to be ‘FD&C’ certified may be used to provide coloring to the product and are within the purview of the present invention. Suitable colorants include natural colorants i.e., pigments and dyes obtained from mineral, plant, and animal sources. Examples of natural colorants include red ferric oxide, yellow ferric oxide, annattenes, alizarin, indigo, rutin, quercetin, and the like. Synthetic colorants may also be used, which is typically an FD&C or D&C dye, e.g., an approved dye selected from the so-called ‘coal-tar’ dyes, such as a nitroso dye, a nitro dye, an azo dye, an oxazine, a thiazine, a pyrazolone, a xanthene, an indigoid, an anthraquinone, an acridine, a rosaniline, a phthalein, a quinoline, or a ‘lake’ thereof, i.e. an aluminum or calcium salt thereof. Particularly preferred colorants are food colorants in the ‘GRAS’ (Generally Regarded as Safe) category.

In another embodiment, the composition according to the present invention comprises tadalafil and at least one water soluble polymer in the ratio about 50:1 to about 1:50. In another embodiment, the composition according to the present invention comprises tadalafil in the range of 1 to 50 mg.

In another embodiment, the present invention relates, to a process for preparing a pharmaceutical composition comprising tadalafil or a pharmaceutically acceptable salt thereof and one or more pharmaceutically acceptable excipients comprising the step of heating a mixture of tadalafil and one or more pharmaceutically acceptable excipients and formulating into a suitable dosage form. In an embodiment, the heating can be done using any equipment known in the art such as, but not limited to, ordinary stove, homogenizer or a hot melt extruder. When an ordinary stove is used the mixture is heated for about 1 hour to about 3 hours, or about 1.5 hours to about 2.5 hours, or about 2 hours, to achieve substantially complete melting of the excipients. When a homogenizer is used, the heating time can even be much less than one hour and when an extruder is used, the heating time can be about 1 minute to about 5 minutes. In another embodiment of the present invention, heating can advantageously also take place in an extruder, so that a homogeneous extrudate of active substance and excipient or solvent is obtained. Extrusion according to the invention preferably takes place at a discharge pressure from about 10 bar to about 100 bar, preferably from about 20 bar to about 100 bar, in particular from about 20 bar to about 50 bar. In particular, it is advantageous if the extruder has a temperature gradient from about 20° C. (inlet temperature) to about 95° C. (outlet temperature).

In another embodiment, the present invention relates to a process for preparing a pharmaceutical composition comprising tadalafil, which comprises the steps of:

(i) blending tadalafil with one or more pharmaceutically acceptable excipients,

(ii) melt extruding the material of step (i) using a hot melt extruder by maintaining the temperature below 100° C. in different zones of the said melt extruder,

(iii) blending the extrudates of step (ii) with one or more pharmaceutically acceptable excipients,

(iv) formulating the blend of step (iii) into a suitable dosage form, and

(v) optionally coating the dosage form thus obtained in step (iv).

In another embodiment, the present invention relates to a process for preparing a pharmaceutical composition comprising tadalafil, which comprises the steps of:

(i) blending tadalafil having particle size D₉₀ more than about 40 microns, with one or more pharmaceutically acceptable excipients,

(ii) melt extruding the material of step (i) using a hot melt extruder by maintaining the temperature below 100° C. in different zones of the said melt extruder,

(iii) blending the extrudates of step (ii) with one or more pharmaceutically acceptable excipients,

(iv) formulating the blend of step (iii) into a suitable dosage form, and

(v) optionally coating the dosage form thus obtained in step (iv).

In another embodiment, the present invention relates to a process for preparing a pharmaceutical composition comprising tadalafil, which comprises the steps of:

(i) blending tadalafil having particle size D90 in the range of between about 42 microns to less than about 200 microns, with one or more pharmaceutically acceptable excipients,

(ii) melt extruding the material of step (i) using a hot melt extruder by maintaining the temperature below 95° C. in different zones of the said melt extruder,

(iii) blending the extrudates of step (ii) with one or more pharmaceutically acceptable excipients,

(iv) formulating the blend of step (iii) into a suitable dosage form, and

(v) optionally coating the dosage form thus obtained in step (iv).

In another embodiment, the present invention relates to a process for preparing a pharmaceutical composition comprising tadalafil, which comprises the steps of:

(i) blending tadalafil having particle size D90 in the range of between about 42 microns to less than about 200 microns, with one or more pharmaceutically acceptable excipients,

(ii) melt extruding the material of step (i) using a hot melt extruder by maintaining the temperature below 95° C. in different zones of the said melt extruder,

(iii) blending the extrudates of step (ii) with one or more pharmaceutically acceptable excipients,

(iv) compressing the blend of step (iii) into tablets or filling into capsules, and

(v) optionally coating the compressed tablets thus obtained in step (iv).

In another embodiment, the present invention relates to a process for preparing a pharmaceutical composition comprising tadalafil, which comprises the steps of:

(i) blending tadalafil having particle size D90 in the range of between about 42 microns to less than about 200 microns, at least one water soluble polymer, and one or more pharmaceutically acceptable excipients,

(ii) adding a surfactant to the blend of step (i),

(iii) melt extruding the material of step (ii) using a hot melt extruder by maintaining the temperature below 95° C. in different zones of the said melt extruder,

(iv) blending the extrudates of step (iii) with one or more pharmaceutically acceptable excipients,

(v) compressing the blend of step (iv) into tablets, and

(vi) optionally film coating the compressed tablets obtained in step (v).

In another embodiment, the present invention relates to a process for preparing a pharmaceutical composition comprising tadalafil, which comprises the steps of:

(i) blending tadalafil having particle size D90 in the range of between about 42 microns to less than about 200 microns, copovidone and one or more pharmaceutically acceptable excipients,

(ii) adding polyoxyl 40 hydrogenated castor oil to blend of step (i),

(iii) melt extruding the material of step (ii) using a hot melt extruder by maintaining the temperature below 95° C. in different zones of the said melt extruder,

(iv) blending the extrudates of step (iii) with one or more pharmaceutically acceptable excipients,

(v) compressing the blend of step (iv) into tablets, and

(vi) optionally film coating the compressed tablets obtained in step (v).

In another embodiment, the composition comprising tadalafil according to the present invention comprises water content in the range of 1-10%.

In another embodiment, the composition comprising tadalafil according to the present invention can be used for the treatment of sexual dysfunction such as male erectile dysfunction or female sexual arousal disorder; benign prostatic hyperplasia and pulmonary hypertension. In another embodiment, the present invention further relates to a method of treating sexual dysfunction and/or benign prostatic hyperplasia and/or pulmonary hypertension by administering a pharmaceutical composition comprising tadalafil or a pharmaceutically acceptable salt thereof and one or more pharmaceutically acceptable excipients. In yet another embodiment, the present invention relates to use of tadalafil or a pharmaceutically acceptable salt thereof in the preparation of a medicament for the prevention or treatment of sexual dysfunction and/or benign prostatic hyperplasia and/or pulmonary hypertension. In an embodiment, the present invention relates to tadalafil or a pharmaceutically acceptable salt thereof for use in treating sexual dysfunction and/or benign prostatic hyperplasia and/or pulmonary hypertension.

The following examples further exemplify the invention and are not intended to limit the scope of the invention. It is obvious to those skilled in the art to find out the composition for other dosage forms and substitute the equivalent excipients as described in this specification or with the one known to the industry.

### Examples 1-5

| | Example-1 | Example-2 | Example-3 | Example-4 | Example-5 |
|---|---|---|---|---|---|
| Ingredients | Qty (% w/w) | Qty (mg/tablet) | Qty (mg/tablet) | Qty (mg/tablet) | Qty (mg/tablet) |
| Tadalafil | 1-20% | 20.0 | 5.00 | 2.50 | 20.0 |
| Copovidone | 1-20% | — | 3.75 | — | 20.0 |
| Hydroxypropyl methylcellulose | — | 15.0 | — | — | — |
| Polyethylene glycol 400 | — | — | — | — | 4.0 |
| Hydroxypropyl cellulose | — | — | — | 20.0 | — |
| Polyoxyl 40 Hydrogenated | 0.001-5% | — | 2.25 | — | — |
| Castor Oil | | | | | |
| Polysorbate 80 | — | 9.0 | — | — | — |
| Sodium lauryl sulphate | — | — | — | 4.0 | — |
| Lactose monohydrate | 5-95% | — | 5.00 | — | — |
| Colloidal silicon dioxide | 0.01-5% | — | 0.50 | — | — |
| Talc | — | 2.0 | — | — | — |
| Dibasic calcium phosphate | — | — | — | 140.5 | — |
| Lactose monohydrate | 5-95% | 150.0 | 47.50 | 158.0 | 150.0 |
| Microcrystalline cellulose | 5-95% | 110.0 | 13.00 | — | 158.0 |
| Croscarmellose Sodium | 1-30% | 36.0 | 9.00 | 40.0 | — |
| Sodium starch glycolate | — | — | — | — | 40.0 |
| Colloidal Silicon dioxide | 0.01-5% | 4.0 | 1.00 | 5.0 | — |
| Talc | — | — | — | — | 4.0 |
| Magnesium stearate | 0.01-5% | 4.0 | 1.00 | 5.0 | — |
| Zinc stearate | — | — | — | — | 4.0 |
| Film Coating | | | | | |
| Opadry II ® yellow | — | 358.0 | 90.20 | — | — |
| Opadry II ® purple | — | — | — | 384.0 | — |
| Purified water* | — | q.s. | q.s. | q.s. | — |

Manufacturing Procedure (Example-1):
i). Tadalafil, copovidone, lactose monohydrate and colloidal silicon dioxide were sifted and blended together,
ii). Polyoxyl 40 hydrogenated castor oil was melted and added to the blend of step (i),
iii). The material of step (ii) was melt extruded using a hot melt extruder by maintaining the temperature below 100° C. in different zones of the melt extruder, and the extruded mass was milled and sifted,
iv). The extrudates of step (iii) were blended with lactose monohydrate, microcrystalline cellulose, croscarmellose sodium and colloidal silicon dioxide,
v). The blend of step (iv) was lubricated with magnesium stearate and compressed to form tablets.

Coating Material:
vi). The coating material was prepared by dispersing specified amount of Opadry II® in purified water.
vii). The coating material of step (vi) was sprayed on to the tablets of step (v) to obtain the coated tablets having a desired weight gain.

The compositions given in Examples 2 to 5 were prepared using the similar procedure described in Example 1.

Table-1 given below shows the comparative dissolution profile of tadalafil tablets according to the present invention (Examples 1-5) and Cialis® Tablets carried out in 1000 ml medium (water+0.5% sodium lauryl sulphate) using Apparatus USP II (Paddle), at 50 rpm speed.

TABLE 1

| | % Drug released | | | | | |
|---|---|---|---|---|---|---|
| Time in | Exam- | Exam- | Example | Example | Exam- | Cialis ® |
| mins. | ple 1 | ple 2 | 3 | 4 | ple 5 | Tablets |
| 10 | 83 | 80 | 82 | 77 | 81 | 67 |
| 15 | 94 | 89 | 85 | 78 | 84 | 80 |
| 30 | 103 | 96 | 91 | 81 | 89 | 93 |
| 45 | 105 | 97 | 93 | 83 | 90 | 96 |
| 60 | 105 | — | 95 | 84 | 92 | 97 |

The comparative dissolution data shows that the compositions of the present invention have a comparable dissolution with regard to the marketed product Cialis® Tablets.

We claim:

1. A pharmaceutical composition comprising tadalafil or a pharmaceutically acceptable salt thereof having particle size D₉₀ more than 40 microns, and one or more pharmaceutically acceptable excipients.

2. The composition according to claim 1, comprising tadalafil having particle size D90 in the range of between about 42 microns to less that about 200 microns.

3. The composition according to claim 1, wherein the composition is prepared by melt extrusion process.

4. The composition according to claim 3, wherein the composition is prepared by melt extrusion process by maintaining the temperature below about 100° C. in different zones of the melt extruder.

5. The composition according to claim 3, wherein the composition is prepared by melt extrusion process by maintaining the temperature between about 40° C. to about 95° C. in different zones of the melt extruder.

6. The composition according to claim 1, wherein the pharmaceutically acceptable excipients are selected from group comprising water soluble polymers, diluents or fillers, binders, disintegrants, antioxidants, sugars, lubricants, glidants, compression aids, colors, sweeteners, preservatives, surfactants, suspending agents, dispersing agents, film formers, flavors and printing inks, and mixtures thereof.

7. The composition according to claim 6, wherein the water soluble polymers are selected from group comprising homopolymers and copolymers of N-vinyl lactams, especially homopolymers and copolymers of N-vinyl pyrrolidone, methyl cellulose, hydroxypropyl cellulose, hydroxypropyl methyl cellulose, cellulose phthalates or succinates, cellulose acetate phthalate hydroxypropyl methylcelluose phthalate, polyethylene oxide, polyacrylates, methyl methacrylate, butyl methacrylate, polyvinyl caprolactam-polyvinyl acetate-polyethylene glycol graft, polyethylene glycol, and mixtures thereof.

8. The composition according to claim 6, wherein the diluents are selected from group comprising sugars; sugar alcohols; co-processed mixture of starch and lactose; co-processed mixture of microcrystalline cellulose and lactose; starch; corn starch; modified starches; pregelatinized starch; dibasic calcium phosphate; tribasic calcium phosphate; powdered cellulose; microcrystalline cellulose; silicified microcrystalline cellulose; and mixtures thereof.

9. The composition according to claim 6, wherein the binders are selected from group comprising cellulose and its derivatives; starch and its derivatives; polyvinyl alcohol; polyvinyl alcohol based compositions; hydrocolloids; sugars; polyvinylpyrrolidone, copovidone; methacrylic acid copolymers; and mixtures thereof.

10. The composition according to claim 6, wherein the disintegrants are selected from group comprising water swellable substances; cross-linked polyvinylpyrrolidone; cross-linked sodium carboxymethylcellulose; cross-linked calcium carboxymethylcellulose; sodium carboxymethylcellulose; calcium carboxymethylcellulose; microcrystalline cellulose; sodium starch glycolate; ion-exchange resins; starch and modified starches; formalin-casein; alginates; gums; and mixtures thereof.

11. The composition according to claim 6, wherein the surfactants are selected from group comprising polyoxyethylene alkyl aryl ethers; polyethylene glycol; polyethylene glycol fatty acid esters; polyoxyethylene sorbitan fatty acid ester; sorbitan fatty acid mono esters; polyoxyethylene castor oil derivates; sodium lauryl sulphate; sodium dioctyl sulfosuccinate; lecithin; stearylic alcohol; cetostearyl alcohol; cholesterol; polyoxyethylene ricin oil; polyoxyethylene fatty acid glycerides; poloxamer; and mixtures thereof.

12. The composition according to claim 6, wherein the lubricants are selected from group comprising calcium stearate, glyceryl behenate, magnesium stearate, mineral oil, polyethylene glycol, sodium stearyl fumarate, stearic acid, fumaric acid, talc, vegetable oil, zinc stearate, and mixtures thereof.

13. The composition according to claim 1, wherein the composition comprises tadalafil and at least one water soluble polymer in the ratio of about 50:1 to about 1:50.

14. A process for preparing a pharmaceutical composition comprising tadalafil according to claim 1, which comprises the steps of:
(i) blending tadalafil with one or more pharmaceutically acceptable excipients,
(ii) melt extruding the material of step (i) using a hot melt extruder by maintaining the temperature below 100° C. in different zones of the said melt extruder,
(iii) blending the extrudates of step (ii) with one or more pharmaceutically acceptable excipients,
(iv) formulating the blend of step (iii) into a suitable dosage form, and
(v) optionally coating the dosage form thus obtained in step (iv).

15. The process of claim 14 wherein the blended tadalafil of step (i) has a particle size D90 more than about 40 microns.

16. The process of claim 14 wherein the blended tadalafil of step (i) has a particle size D90 in the range of between about 42 microns to less than about 200 microns.

17. The process of claim 16 wherein the suitable dosage form of step (iv) is a tablet.

18. The process of claim 16 wherein in step (i) at least one water soluble polymer is blended with tadalafil and a surfactant is finally added to the blend.

19. The process of claim 16 wherein in step (i) copovidone is blended with tadalafil and polyoxyl 40 hydrogenated castor oil is finally added to the blend.

20. (canceled)

21. A method of treating sexual dysfunction and/or benign prostatic hyperplasia and/or pulmonary hypertension by administering a pharmaceutical composition according to claim 1 comprising tadalafil or a pharmaceutically acceptable salt thereof and one or more pharmaceutically acceptable excipients.

22. (canceled)

23. (canceled)
